(19) United States
(12) Patent Application Publication
(10) Pub. No.: US 2021/0329956 A1
(43) Pub. Date: Oct. 28, 2021

(54) Thickened composition for dysphagia patients

(71) Applicant: N.V. Nutricia, Zoetermeer (NL)

(72) Inventors: Mirian Lansink, Utrecht (NL); Sonia Guida, Utrecht (NL); Pedro Clavé Civit, Barcelona (ES)

(73) Assignee: N.V. Nutricia, Zoetermeer (NL)

(21) Appl. No.: 17/270,928

(22) PCT Filed: Aug. 30, 2018
(86) PCT No.: PCT/NL2018/050558

§ 371 Date: Feb. 24, 2021

(51) Int. Cl. A23L 29/238; A23L 29/244; A23L 29/25

(52) U.S. Cl. CPC A23L 29/238; A23L 29/25; A23L 29/244

(57) ABSTRACT

The present invention relates to nutrition for dysphagia. More specifically it relates to a thickened composition for use in decreasing the laryngeal vestibule closure (LVC) time and/or decreasing the duration of swallowing response in a dysphagia patient, wherein the thickened composition comprises a thickening agent comprising galactomannan polysaccharide. The invention also pertains to a thickening agent for use in decreasing the laryngeal vestibule closure (LVC) time and/or decreasing the duration of swallowing response in a dysphagia patient who is consuming a composition to which the thickening agent comprising galactomannan polysaccharide has been added.

### FIELD OF THE INVENTION

The present invention relates to nutrition for dysphagia patients and specifically to nutrition for decreasing the laryngeal vestibule closure (LVC) time and/or decreasing the duration of swallowing response in said patients.

### BACKGROUND OF THE INVENTION

Dysphagia patients experience difficulty in swallowing properly. These patients often have a loss of sense in the oral cavity and pharynx and lack a proper swallowing reflex. Furthermore, it is often very difficult for these patients to handle low viscosity food as it may enter into the trachea. As a result these patients need to resort to specialized drinks and foods modified such that they do not enter into the trachea. Usually these specialized drinks and foods have an increased viscosity, whereby the exact viscosity may vary depending on the severity of the swallowing problem.

Dysphagia patients generally lack proper muscle control and coordination to properly seal their larynx or they lack the ability to properly propel the entire bolus of food and/or beverage to the stomach. Dysphagia patients can be afraid to consume foodstuffs that appear too thin to them, because they may fear to aspirate and/or choke. This fear often causes a reluctance to eat or drink and may lead to dehydration and mal-nutrition. It is therefore crucial that the foodstuffs dysphagia patients consume have the proper sensorial properties, such as viscosity, when they judge the food visually and when they feel it in their mouth during consumption. In addition it is imperative that the food has those properties ensuring that swallowing is more easy and the risk of aspiration is reduced.

Thus thickening foods and/or beverages for humans afflicted with dysphagia is a common method of managing dysphagia. Thickening provides better bolus control, greater oral stimulation, and when swallowed, it helps prevent undesired and potentially fatal aspiration of fluids into the lungs.

Traditionally starch is used to thicken beverages of dysphagia patients. Nowadays, gums, such as xanthan gum, tara gum or locust bean gum, are more common. Several specialized products for dysphagia patients are available for thickening liquids and foods. For example, a thickening product for dysphagia patients comprising xanthan gum is known from WO2004/069179 or from Rofes et al. (Aliment Pharmacol Ther 2014; 39: 1169-1179). Compositions comprising xanthan gum and taragum or locust bean gum are known from JP2000191553. EP1841332 discloses thickened nutritional products for dysphagia patients comprising xanthan gum or methylcellulose along with starch, tara gum and maltodextrin.

All of the above mentioned products have an adapted viscosity such that they can more safely be consumed by dysphagia patients. Hence the properties of the product compensate for the swallowing problem that dysphagia patients have. There is currently a trend to move to strategies that not only compensate for the swallowing problem of dysphagia patients, but also provide for (at least a partial) recovery. For example, as described by Cabib et al. in (Ann. N.Y. Acad. Sci. (2016): 1-18), patients suffering from oropharyngeal dysphagia greatly benefit from compensatory treatment supplemented with a treatment that promotes brain plasticity such that the swallow function is recovered and/or the brain-related swallowing dysfunction is improved.

There is now a need for thickened products for dysphagia patients that not only compensate for the swallowing problem, but also provide for at least a partial recovery of the swallowing function and/or at least an improvement of the brain-related swallowing dysfunction. Hence there is a need for a thickened product having an increased viscosity that helps dysphagia patients swallow better.

### SUMMARY OF THE INVENTION

The inventors have surprisingly found that a composition thickened with galactomannan polysaccharide not only compensates for the swallowing problem in dysphagia patients but also invokes a partial recovery of the swallowing function, at least while consuming said thickened composition. Using imaging techniques the inventors have now shown for the first time that dysphagia patents consuming a composition thickened with galactomannan polysaccharide show a decreased laryngeal vestibule closure (LVC) time and a decreased duration of the swallowing response. Both the decreased LVC time and the decreased duration of the swallowing response are indications of recovery of the swallowing function and help to swallow better. Absent such effects observed for similarly viscous, thickened compositions comprising xanthan gum, these advantageous effects could not be attributed to viscosity only. Hence the composition thickened with galactomannan polysaccharide compensates for the swallowing problem and invokes a partial recovery of the swallowing function.

The invention therefore encompasses a thickening agent and a thickened composition for use in decreasing the LVC time and/or decreasing the duration of swallowing response in a dysphagia patient, wherein the thickening agent is added to obtain the thickened composition, and wherein the thickening agent comprises galactomannan polysaccharide. Preferably the thickened composition is a thickened liquid composition. In a further preferred embodiment the thickened liquid composition has a viscosity of 20-3000 mPa·s, preferably of 80-800 mPa·s, for instance determined using cone-plate viscosity measurements at 50 s-1 and 20° C. The thickening agent preferably comprises fenugreek gum, guar gum, tara gum, locust bean gum and/or cassia gum. In an embodiment the thickening agent may further comprise additional thickeners such as xanthan gum.

The thickening agent may be a powder that can be added to a composition that needs to be thickened. Said powder may suitably comprise other ingredients, such as maltodextrin. The powdered thickening agent can be added to a composition in order to get a thickened composition. Further details are provided in the detailed description.

The thickened composition for use according to the invention may be any thickened nutritional composition such as a drink, simply water, meal replacement or nutritional supplement which fits in the dietary management of a dysphagia patient. In one embodiment, the thickened nutritional composition further comprises 25-40 en % of fats, 40-60 en % of carbohydrates and 10-25 en % of proteins. Alternatively or additionally the thickened composition has an energy density in the range of 10-300 kcal/100 ml.

### DETAILED DESCRIPTION

In a first aspect, the invention encompasses a thickening agent for use in decreasing the laryngeal vestibule closure (LVC) time and/or decreasing the duration of swallowing response in a dysphagia patient who is consuming a thickened composition comprising a thickening agent comprising galactomannan polysaccharide i.e. a composition to which a thickening agent comprising galactomannan polysaccharide has been added (prior to administration). The invention may also be worded as the use of a thickening agent for the manufacture of a thickened composition for the treatment of a dysphagia patient, wherein the treatment comprises decreasing the laryngeal vestibule closure (LVC) time and/or decreasing the duration of swallowing response in said dysphagia patient who is consuming a thickened composition comprising a thickening agent comprising galactomannan polysaccharide i.e. a composition to which a thickening agent comprising galactomannan polysaccharide has been added (prior to administration). Alternatively, the invention can be worded as a method for decreasing the laryngeal vestibule closure (LVC) time and/or decreasing the duration of swallowing response in a dysphagia patient, wherein the method comprises adding a thickening agent to a composition, to obtain a thickened composition, and administering the thickened composition comprising galactomannan polysaccharide to the dysphagia patient.

The thickening agent is preferably added to a ready-to-use nutritional product. In a preferred embodiment, the thickening agent is added to the composition (i.e. the ready-to-use nutritional product), to obtain a thickened nutritional product, within 15 minutes before administration. The preparation may be carried out by the patient or by medical staff, nurse practioners or care-takers.

In a second aspect, the invention encompasses a thickened composition for use in decreasing the laryngeal vestibule closure (LVC) time and/or decreasing the duration of swallowing response in a dysphagia patient, wherein the thickened composition comprises a thickening agent comprising galactomannan polysaccharide or wherein the thickened composition is prepared by adding a thickening agent comprising galactomannan polysaccharide to a composition. The invention may also be worded as the use of a thickened composition for the manufacture of a product for the treatment of a dysphagia patient, wherein the treatment comprises decreasing the laryngeal vestibule closure (LVC) time and/or decreasing the duration of swallowing response in said dysphagia patient and wherein the thickened composition comprises a thickening agent comprising galactomannan polysaccharide or wherein the thickened composition is prepared by adding a thickening agent comprising galactomannan polysaccharide to a composition. Alternatively, the invention can be worded as a method for decreasing the laryngeal vestibule closure (LVC) time and/or decreasing the duration of swallowing response in a dysphagia patient, wherein the method comprises administering a thickened composition comprising galactomannan polysaccharide or a thickened composition prepared by adding a thickening agent comprising galactomannan polysaccharide to a composition to the dysphagia patient.

The dysphagia patient is a mammal, preferably a human being.

The composition to which the thickening agent is added as well as the thickened composition comprising the thickening agent are preferably characterized being a nutritional composition.

The use according to the invention is a medical use. The dysphagia diet is typically determined by medical consults, speech and language therapists, dietitians and/or nursing staff. The thickening agent is typically added to the composition, to obtain a thickened composition having specific consistency or viscosity, in predetermined amounts.

However, if desired the composition of the invention can also be used in a non-medical method. In this case the invention may be worded as a method for decreasing the laryngeal vestibule closure (LVC) time and/or decreasing the duration of swallowing response in a mammal, wherein the method comprises adding a thickening agent to a composition, to obtain a thickened composition, and administering the thickened composition to the mammal. In a preferred embodiment the mammal is a human being. In a further preferred embodiment the human being is a human being suffering from swallowing problems and in an even further preferred embodiment said human being is not diagnosed with dysphagia.

The treatment may involve a step of monitoring the LVC time.

### Thickening

In the context of the invention a thickening agent refers to an agent that can be added to a composition causing an increase in thickness or viscosity of the composition. A composition comprising a thickening agent and having an increased viscosity may be referred to as a thickened composition. The thickened composition without a thickener may be referred to as the unthickened composition. The thickened composition has an increased viscosity compared to the unthickened composition. This is a consequence of the addition of the thickening agent in predetermined amounts.

Preferably the thickening agent increases the viscosity of the unthickened composition with at least 10 mPa·s, preferably 20 mPa·s, more preferably at least 30 mPa·s, even more preferably at least 40 mPa·s, even more preferably at least 50 mPa·s, even more preferably at least 75 mPa·s, even more preferably at least 100 mPa·s, even more preferably at least 150 mPa·s, even more preferably at least 250 mPa·s, even more preferably at least 500 mPa·s, even more preferably at least 750 mPa·s. The increase in viscosity can be determined using any suitable method for measuring viscosity. Preferably the viscosity is determined using an Anton Paar Physica Rheometer MCR 301 (Anton Paar GmbH, Graz, Austria) with cone-plate geometry (CP50-1/PC) after 15 minutes at a shear rate of 50 and at 20° C., comparing the composition before and after adding the thickening agent (i.e. the unthickened vs. the thickened composition).

According to the invention the thickening agent comprises a galactomannan polysaccharide. Galactomannan polysaccharides may refer to a polysaccharide having a mannose backbone with galactose side groups. The mannose backbone may comprise (1-4)-linked beta-D-mannopyranose. The galactose side groups may be situated at the 6-positions of the mannose and may be linked to alpha-D-galactose. Known thickening agents comprising galactomannan polysaccharide include fenugreek gum, guar gum, tara gum, locust bean gum and cassia gum. Preferably, the thickening agent comprises guar gum and/or tara gum, most preferably at least guar gum.

It is preferred that the thickened composition is a thickened nutritional liquid product, and the unthickened composition is an unthickened nutritional liquid product. In this context a nutritional product refers to any product that is suitable for use as nutrition. As such a nutritional product may refer to any food product. Here a food product may comprise fats, carbohydrates, proteins, fibres, vitamins, minerals and/or other nutrients. A food product may also refer to a beverage, which may be simply water, but may also be thee, coffee, milk, apple juice or any other beverage suitable for consumption. In this context a liquid refers to a fluid that can flow and that will adapt its shape to its container. The thickness or viscosity of the liquid determines the time a liquid needs to adapt to the shape of the container. Thin liquids are keen to flow and adapt quickly to the shape of the container. Thick liquids are at least somewhat resistant to flowing and might even appear solid. Notably pureed foods are also considered a liquid provided they adapt their shape to the container retaining them. Furthermore, as is custom in the art, a liquid also refers to a liquid with particles or chunks. Hence a soup is considered a liquid even if it comprises e.g. croutons and/or noodles.

### Thickened Composition

The thickened composition for use according to the current invention has a viscosity of at least 20 mPa·s, preferably 30 mPa·s, more preferably at least 40 mPa·s, even more preferably at least 50 mPa·s, even more preferably at least 60 mPa·s, even more preferably at least 70 mPa·s, even more preferably at least 80 mPa·s, even more preferably at least 90 mPa·s, even more preferably at least 100 mPa·s, even more preferably at least 125 mPa·s, even more preferably at least 150 mPa·s and even more preferably at least 200 mPa·s. Alternatively worded the composition for use according to the invention has a viscosity of 20-3000 mPa·s, more preferably of 30-2500 mPa·s, more preferably of 40-2250 mPa·s, more preferably of 50-2000 mPa·s, more preferably of 60-1750 mPa·s, more preferably of 70-1500 mPa·s, more preferably of 75-1000 mPa·s and more preferably of 80-800 mPa·s. The viscosity can be determined using cone-plate viscosity measurements at 50 s-1 and 20° C.

In the art there exist different classification systems for compositions suitable in dysphagia treatment, and these thickened compositions for use in the treatment of dysphagia patients are often denoted according to their consistency.

In one embodiment, the thickened compositions comply with i) syrup, nectar or nectar-thick, ii) custard, honey or honey-thick and iii) pudding or spoon-thick consistency, as determined using the Texture Modification Tables for use by Speech and Language Therapists and Dietitians. Here the terms syrup, custard and pudding consistency are used according to the general agreement proposed by the British Dietetic Association and Royal College of Speech and Language Therapists (see National Descriptors for Texture Modification in Adults. BDA, 2002). Briefly, a syrup or nectar consistency implies that the product (i) can be drunk through a straw, (ii) can be drunk from a cup if advised or preferred and (iii) leaves a thin coat on the back of a spoon. A custard or honey consistency implies that the product (i) cannot be drunk through a straw, (ii) can be drunk from a cup and (iii) leaves a thick coat on the back of a spoon. A pudding or spoon-thick consistency implies that the product (i) cannot be drunk through a straw, (ii) cannot be drunk from a cup and (iii) needs to be taken with a spoon.

The thickened composition for use according to the current invention preferably has a syrup (or nectar), custard (or honey) or pudding (or spoon thick) consistency. More preferably the thickened composition for use has syrup or custard consistency. Thus for the current invention and throughout this document a liquid may refer to a fluid having syrup, custard or pudding consistency. The thickened composition for use according to the current invention is a thickened liquid product having syrup, custard or pudding consistency. Preferably the thickened composition for use according to the current invention is a thickened liquid product having syrup or custard consistency.

In another embodiment, the thickened compositions have IDDSI levels 1-5, preferably IDDSI levels 2-6, more preferably IDDSI levels 2-5 and more preferably IDDSI levels 2-4, as determined by the standardized IDDSI (International Dysphagia Diet Standardization Initiative) which involves a continuum of 8 levels (0-7) describing the thickness of food compositions. Reference is made to Cichero J A, Lam P, Steele C M, Hanson B, Chen J, Dantas R O, Duivestein J, Kayashita J, Lecko C, Murray J, Pillay M, Riquelme L, Stanschus S. “Development of International Terminology and Definitions for Texture-Modified Foods and Thickened Fluids Used in Dysphagia Management: The IDDSI Framework” Dysphagia. 2017 April; 32(2):293-314, its contents herewith incorporated by reference. The IDDS classification is as follows:
0 thin:
   flows like water, can be drunk from a cup or through a teat/nipple or straw.
1 slightly thick:
   thicker than water, requires a little more effort to drink than thin liquids, flows through a teat/nipple or a straw.
   corresponds to 30 mPa·s
2 mildly thick:
   flows off a spoon, sippable, effort required to drink through a straw.
3 liquidised/moderately thick
   Can be drunk from a cup, cannot be eaten with a fork, no chewing required.
4 pureed/extremely thick:
   Usually eaten with a spoon, cannot be drunk from a cup, does not require chewing
5 minced and moist
   Can be eaten with a fork or spoon
   Can be scooped
   Minimal chewing required
6 Soft and bite-sized
   Can be eaten with a fork, spoon or chopstick
   Chewing is required before swallowing
   Soft tender and moist throughout
7 Regular
   Normal everyday foods of various textures that are developmentally and age appropriate

### Thickening Agents

In order to arrive at the preferred thickened compositions for use in the treatment of dysphagia patients, a thickening agent is used to be added to a nutritional composition, to arrive at the desired viscosity. The amount of thickening agent is determined with or by speech and language therapists, nursing staff and/or dietitians.

The thickening agent, preferably a powder with preferably less than 15 wt % (more preferably less than 10 wt %) water, which agent comprises galactomannan polysaccharide, preferably in an amount of 0.025-5.0 wt %, more preferably 0.05-4.0 wt %, more preferably 0.1-3.0 wt %, more preferably 0.15-2.0 wt %, most preferably 0.15-1.0 wt %, most preferably 0.2-1.0 wt % of galactomannan polysaccharide, based on total weight of the agent. Even more preferred the thickening agent comprises 0.025-5.0 wt %, more preferably 0.05-4.0 wt %, more preferably 0.1-3.0 wt %, more preferably 0.15-2.0 wt %, most preferably 0.15-1.0 wt %, most preferably 0.2-1.0 wt % of guar gum, based on total weight of the agent.

The thickening agent may suitably further comprise further thickeners as known in the art. Preferably, the thickening agent further comprises xanthan gum. Xanthan gum may refer to the well-known food thickener that can be obtained through fermentation of sugar by Xanthomonas campestris. Preferably the xanthan gum used for the invention is a purified xanthan gum that is suitable for use as a clear or transparent thickener. Preferably the thickening agent comprises 20-50 wt %, more preferably 25-40 wt %, more preferably 25-35 wt % of xanthan gum, based on the total weight of the agent.

The thickening agent according to the invention may hence comprises xanthan gum and galactomannan polysaccharide. Preferably the weight ratio of xanthan gum to galactomannan polysaccharide is in the range of 5-750, preferably 6-500, more preferably 10-400, even more preferably 20-300, more preferably 30-250, most preferably 40-200, particularly 50-150, most preferably 60-140.

In a most preferred embodiment, the thickening agent comprises xanthan gum and guar gum, with a weight ratio of xanthan gum to guar gum in the range of 5-750, preferably 6-500, more preferably 10-400, even more preferably 20-300, more preferably 30-250, most preferably 40-200, particularly 50-150, most preferably 60-140. In addition, the thickening agent may comprise 20-50 wt. % of xanthan gum and 0.05-5.0 wt. % of guar gum, preferably 20-50 wt. % of xanthan gum and 0.05-4.0 wt. % of guar gum, even more preferably 25-40 wt. % of xanthan gum and 0.1-3 wt. % of guar gum, more preferably 25-40 wt % xanthan gum and 0.15-2.0 wt % guar gum, most preferably 25-35 wt % xanthan gum and 0.15-1.0 wt % guar gum, particularly 25-35 wt % xanthan gum and 0.2-1.0 wt % guar gum.

Furthermore the thickening agent may comprise additional ingredients such as 20-80 wt. %, preferably 30-75 wt. %, more preferably 40-70 wt. % and even more preferably 60-70 wt. % of maltodextrin, based on total weight of the agent. Preferably the thickening agent comprises 20-80 wt. %, preferably 30-75 wt. %, more preferably 40-70 wt. % and even more preferably 60-70 wt. % of maize maltodextrin, based on total weight of the agent.

The thickening agent is preferably provided in packaged form, with instructions for addition to compositions for the dietary management of dysphagia patients. The packaged agent may be provided together with a level scoop.

### Application

The thickened composition according to the invention is for use in the treatment of dysphagia patients, i.e. patients suffering from dysphagia. Dysphagia is a gastrointestinal motility disorder and relates to a dysfunction of one or more parts of the swallowing apparatus. The swallowing apparatus comprises the mouth including the lips, the tongue, the oral cavity, the pharynx, the airway and the esophagus with the upper/lower sphincter. Dysphagia is recognized by the World Health Organization in the International Statistical Classification of Diseases and Related Health Problems ICD-9 and ICD-10. Symptoms associated with dysphagia are transit problems of food boluses and/or problems with airway protection.

Dysphagia can be classified depending on the cause of the swallowing difficulty. Abnormalities, which are often related to muscle and/or nerve damage, affecting the upper esophageal sphincter, pharynx, larynx, and/or oral cavity, result in oropharyngeal dysphagia. In one embodiment the dysphagia patient is an oropharyngeal dysphagia patient. In a further embodiment the oropharyngeal dysphagia patient is an oropharyngeal dysphagia patient suffering from muscle and/or nerve damage. Said muscle damage may be muscle damage affecting the upper esophageal sphincter, pharynx, larynx, and/or oral cavity. Said nerve damage may be nerve damage affecting the upper esophageal sphincter, pharynx, larynx, and/or oral cavity. Associated symptoms may include transit and/or airway protection problems. Hence in an embodiment of the invention the dysphagia patient is a dysphagia patient suffering from transit and/or airway protection problems. In case transit problems in the esophagus as a result of muscle damage, the term esophageal or obstructive dysphagia is typically used. In one embodiment the dysphagia patient is an esophageal dysphagia patient.

In one embodiment, the cause of the dysphagia is related to the peripheral/central nervous system, to a neurodegenerative disease, the muscular/neuromuscular function, one or more local/structural lesions and/or to drugs. In one embodiment, the cause of dysphagia is selected from stroke, head trauma, Parkinson, a myotrophic lateral sclerosis, multiple sclerosis, Alzheimer, poliomyelitis/postpolio syndrome, polymyositis/dermatomyositis, myasthenia gravis, metabolic myopathy (eg, thyroid myopathy), muscular dystrophies, Kearns-Sayre syndrome, head and neck tumors, surgical resection of the oropharynx/larynx, radiation injury, Zenker diverticulum, extrinsic compression (eg, goiter, cervical osteophyte), and chricopharyngeal achalasia. The dysphagia might be related to aging. Hereby it is understood that aging as such can be a reason for dysphagia. Also any of the previously mentioned causes might be related to aging.

The thickened composition for use according to the invention can effectively be used by all dysphagia patients and is particularly effective for oropharyngeal dysphagia patients such as post stroke oropharyngeal dysphagia patients.

Dysphagia often results in in delayed laryngeal vestibule closure (LVC), wherein the term laryngeal vestibule is used to denote that part of the larynx above the vocal cords. Delayed LVC may cause food to travel down the trachea and can results in aspiration. It is hence an object of the invention to decrease the laryngeal vestibule closure (LVC) time.

An additional or alternative approach to prevent food from traveling down the larynx is to decrease the duration of swallowing. Here the duration of swallowing is defined as the time from the opening of the glossopalatal junction to the opening of the laryngeal vestibule, wherein the glossopalatal junction—which is sometimes referred to as fauces—refers to the passage between the soft palate and the base of tongue.

The thickened composition for use according to the present invention can be thickened in the desired extent. Hence it can be thickened to any viscosity as long as the viscosity is higher than the viscosity of the unthickened product. The increase in viscosity can be controlled by controlling the amount of thickener added.

The thickening agent according to the invention may be in the form of a powder comprising galactomannan polysaccharide that can be added to puréed food, drinks and nutritional supplements. This powder preferably does not change the taste and appearance of the food.

In that case where the patient wants to thicken his/her own food, the patient him/herself can add the thickening agent to the liquid composition. The patient can vary the amount of thickening agent or thickening powder—preferably following the advice of a doctor—such that the thickened nutritional product will have the desired increased viscosity, typically in the form of a syrup, custard or pudding consistency. The thickening agent is preferably added in a predetermined amount between 0.5 and 10 g per 100 ml of the unthickened composition, in order to obtain a thickened composition that complies with the desired consistency, preferably according to the above IDDS or National Descriptors for Texture Modification and/or in order to obtain a thickened composition having the aforementioned viscosity.

Alternatively the nutritional product may be a ready-to-use pre-thickened oral nutritional supplement which has syrup, custard or pudding consistency. This may be a pre-thickened nutritional supplement having syrup, custard or pudding consistency comprising the essential nutrients—such as proteins, fats, carbohydrates, vitamins and minerals—that dysphagia patients often fail to properly ingest. Preferably said pre-thickened composition comprises 25-40 en % of fats, 40-60 en % of carbohydrates and 10-25 en % of proteins. Also the pre-thickened composition preferably has an energy density in the range of 10-300 kcal/100 ml.

The thickened nutritional product according to the invention may be a clear or opaque, thickened nutritional product. Other than consistency and viscosity, the thickening agent preferably does not affect the appearance of the unthickened composition. In this context clear is to be interpreted as transparent or translucent, wherein transparent refers to the property that light is passed without appreciable scattering such that images can be easily seen when looking through the product and translucent refers to the property that light is passed in a scattered and/or diffused manner such that images cannot be recognized when looking through the product. A clear thickened nutritional product is therefore to be understood as a substantially transparently and/or substantially translucently thickened nutritional product, wherein the wording transparently or translucently thickened imply that the thickener substantially does not influence the transparency and/or translucency. Hence the thickening agent does not substantially change the appearance of the nutritional product to be thickened. Hence in that case where the unthickened nutritional product is transparent, the thickened nutritional product can be opaque, transparent or translucent, preferably transparent. When the unthickened nutritional product without thickening is opaque, the thickened nutritional product will also be opaque and will have the same appearance as the unthickened nutritional product.

An appropriate technique to determine the transparency or translucency of a liquid is transmission spectrometry. In this technique a sample of the liquid is held in a container made from a very clear glass or crystal. The container with the sample liquid is put between a light source and a light detector. The light source emits a light beam having a specific frequency. This wavelength should be in the visible spectrum of 380-780 nm. A particularly preferred wavelength is in the range of 400-600 nm. The container preferably has a square or rectangular base such that the light beam entering and exiting the container hits the glass orthogonally. By comparing the intensity of the light hitting the light detector when the container is empty and when the container is filled with the relevant liquid, it is possible to determine the loss of intensity, which is a measure for the transparency. For the current invention the relevant comparison is the light adsorbed by a thickened liquid compared to the light adsorbed by the unthickened liquid.

Similarly an appropriate technique to determine optical properties of an opaque liquid is reflectance spectrometry. The experimental setup is similar as for transmission spectrometry with the notable chance that the light source and the light detector are positioned on the same side of the container such that the light detector measures the reflected light.

The thickening agent according to the invention is a thickening agent that does not substantially change the transparency, translucency and/or opacity of the liquid to which it is added. The loss in light intensity is about equal for the unthickened and the thickened liquid. In an embodiment the translucency of the thickened liquid is at least 50% of the unthickened liquid, more preferably least 60% of the unthickened liquid, even more preferably least 70% of the unthickened liquid, even more preferably least 80% of the unthickened liquid, and even more preferably least 90% of the unthickened liquid. In another embodiment the transparency of the thickened liquid is at least 50% of the unthickened liquid, more preferably least 60% of the unthickened liquid, even more preferably least 70% of the unthickened liquid, even more preferably least 80% of the unthickened liquid, and even more preferably least 90% of the unthickened liquid.

The thickening agent of the invention may suitably be added to the nutritional product to obtain a thickened nutritional product. Preferably said thickened nutritional product is a thickened clear nutritional product or a transparently thickened nutritional product or a translucently thickened product. Alternatively, in case that the nutritional product is opaque the thickened nutritional product is an opaque thickened nutritional product that is visually very similar to the unthickened product.

### Example

To assess and understand the effect of the thickened composition of the invention on swallowing, an experiment was performed. 120 patients of at least 18 years of age with chronic post stroke oropharyngeal dysphagia (post stroke OD) were selected. Inclusion criteria included a diagnosis of stroke, minimum 28 days since the stroke and Clinical signs or symptoms of swallowing dysfunction, based on Eating Assessment Tool (EAT-10) questionnaire, or bedside clinical test or water swallow test, or referral by medical doctor for Video FluoroScopy (VFS)-test, or current use of thickened products. All patients gave their written informed consent.

For every patient, a total of 2 batches of 7 compositions were prepared by mixing an aqueous iodine solution with the appropriate amount of thickening agent. The thickening agent was Nutilis Clear powder with (on average), per 100 g, 0.8 g protein, 57.6 g carbohydrates (of which 46.7 g polysaccharides and 10.9 g sugars), no fat and 28 g dietary fibres and minerals (commercially available with N.V. Nutricia, The Netherlands) and wherein said fiber comprises guar gum and xanthan gum, with a weight ratio of xanthan gum to guar gum of about 60:1 to 100:1. The 2*7 compositions varied in viscosity from thin liquid (iodine solution without thickener; viscosity was about 1 mPa.$), via thickened liquids of 150 mPa·s, 250 mPa·s, 450 mPa·s, 800 mPa·s, 1400 mPa·s to a very thick liquid of 2000 mPa·s. Viscosity was determined using an Anton Paar Physica Rheometer MCR 301 (Anton Paar GmbH, Graz, Austria) with cone-plate geometry (CP50-1/PC) after 15 minutes at a shear rate of 50 and at 20° C.

The swallowing of the patients was monitored using Videofluoroscopy (VFS). This is the gold standard method to evaluate efficacy and safety of swallowing (Clavé et al., Accuracy of the volume-viscosity swallow test for clinical screening of oropharyngeal dysphagia and aspiration. Clin Nutr 2008; 27: 806-15). Using this method the laryngeal vestibule closure time (LVC) and the total duration of swallowing can be determined.

During the study the patients were sitting, with the back resting against the seatback and feet on the ground. The VFS examiner gave a 10 mL bolus of the relevant variety in a syringe to the patient. Each patient swallowed twice a bolus of 10 mL of a product while the swallowing is evaluated using VFS. The order of the products administered is: 1. thin liquid, 2. 2000 mPa·s, 3. 1400 mPa·s, 4. 800 mPa·s, 5. 450 mPa·s, 6. 250 mPa·s, and 7. 150 mPa·s. The results of this study are presented in Table 1.

If a patient would aspirate at the first bolus thin liquid, the second will not be given and the measurements continue with the thickened varieties from the highest viscosity to the lowest viscosity (2000, 1400, 800, 450, 250 and 150 mPa.$). In case the patient would aspirate at the second bolus thin liquid, the measurements would continue with the thickened varieties. When aspiration would occur at any of the measurements with a thickened variety the study stopped for this subject.

The results of this study are summarized in Tables 1 and 2.

TABLE 1
Results on LVC time when patients are given a thin liquid
or a thickened liquid according to the invention.
Results for the comparison of the viscosity with
thin liquid are based on a repeated measures mixed model.
Laryngeal vestibule closure (LVC) time (ms)

| | Thin liquid | 150 mPa · s | 250 mPa · s | |
|---|---|---|---|---|
| | (N = 114) | (N = 114) | (N = 114) | (. . .) |
| Mean | 382.5 | 327.3 | 330.1 | |
| (SD) | (139.1) | (108.2) | (143.4) | |
| p value | | <0.001 | 0.002 | |

| | 450 mPa · s | 800 mPa · s | 1400 mPa · s | 2000 mPa · s |
|---|---|---|---|---|
| | (N = 114) | (N = 114) | (N = 114) | (N = 114) |
| Mean | 304.8 | 303.3 | 300.5 | 300.4 |
| (SD) | (109.6) | (94.7) | (110.0) | (107.8) |
| p value | <0.001 | <0.001 | <0.001 | <0.001 |

TABLE 2
Results on total duration of the swallowing response when
patients are given a thin liquid or a thickened liquid according
to the invention. Results for the comparison of the viscosity with thin
liquid are based on a paired-sample Wilcoxon signed rank test.
Total duration of swallowing response (ms)

| | Thin liquid | 150 mPa · s | 250 mPa · s | |
|---|---|---|---|---|
| | (N = 114) | (N = 114) | (N = 114) | (. . .) |
| Mean | 1020.9 | 947.1 | 998.8 | |
| (SD) | (220.8) | (228.7) | (472.1) | |
| p value | | 0.001 | 0.009 | |

| | 450 mPa · s | 800 mPa · s | 1400 mPa · s | 2000 mPa · s |
|---|---|---|---|---|
| | (N = 114) | (N = 114) | (N = 114) | (N = 114) |
| Mean | 944.1 | 943.1 | 953.5 | 943.2 |
| (SD) | (180.2) | (221.4) | (225.3) | (234.8) |
| p value | <0.001 | <0.001 | <0.001 | <0.001 |

As can be seen from Tables 1 and 2, all thickened liquids of the invention surprisingly show a decreased LVC time and a decreased total duration of swallowing response when administered to post stroke OD patients. This is even more surprising considering that Rofes et al. (Aliment Pharmacol Ther 2014; 39: 1169-1179) previously found in a very similar setup that compositions thickened with only xanthan do not affect LVC time or total duration of swallowing response.

1.-19. (canceled)

20. A method for decreasing the laryngeal vestibule closure (LVC) time and/or decreasing the duration of swallowing response in a dysphagia patient, wherein the method comprises (i) administering a thickened composition comprising galactomannan polysaccharide or (ii) preparing a thickened composition by adding a thickening agent comprising galactomannan polysaccharide to a composition, and administering the thickened composition to the dysphagia patient.

21. The method according to claim 20, wherein the dysphagia patient is an oropharyngeal dysphagia patient.

22. The method according to claim 20, wherein the composition is a liquid composition.

23. The method according to claim 22 wherein the liquid composition has a viscosity of 20-3000 mPa·s, as determined with cone-plate viscosity at a shear rate of 50 s⁻¹ and at 20° C.

24. The method according to claim 20, wherein the thickening agent comprises fenugreek gum, guar gum, tara gum, locust bean gum and/or cassia gum.

25. The method according to claim 20, wherein the thickening agent further comprises xanthan gum.

26. The method according to claim 20, wherein the thickening agent has been added in the form of a powder.

27. The method according to claim 20, wherein the thickening agent comprises maltodextrin.

28. The method according to claim 20, wherein the composition further comprises 25-40 en % of fats, 40-60 en % of carbohydrates and 10-25 en % of proteins.

29. The method according to claim 20, wherein the composition has an energy density in the range of 10-300 kcal/100 ml.

30. The method according to claim 20, wherein the composition is prepared by mixing a liquid composition with the thickening agent.

31. The method according to claim 20, wherein the thickened liquid composition has the same appearance as the liquid composition not comprising the thickening agent.

32. The method according to claim 20, wherein the liquid composition is opaque, transparent and/or translucent.

33. The method according to claim 20, wherein the thickening agent comprises xanthan gum and guar gum, with a weight ratio of xanthan gum to guar gum in the range of 5-750.

34. The method according to claim 33, wherein the thickening agent comprises xanthan gum and guar gum, with a weight ratio of xanthan gum to guar gum in the range of 60-140.

35. The method according to claim 20, wherein the thickening agent comprises 20-50 wt. % of xanthan gum and 0.05-5.0 wt. % of guar gum.

36. The method according to claim 35, wherein the thickening agent comprises 25-35 wt % xanthan gum and 0.2-1.0 wt % guar gum.
